(19) United States
(12) Patent Application Publication
(10) Pub. No.: US 2023/0009293 A1
(43) Pub. Date: Jan. 12, 2023

(54) DEVICE AND METHOD FOR CUTTING BREAST SPECIMEN

(71) Applicant: Hyung Seok Park, Seoul (KR)

(72) Inventors: Hyung Seok PARK, Seoul (KR); Jee A LEE, Seoul (KR)

(73) Assignee: Hyung Seok Park, Seoul (KR)

(21) Appl. No.: 17/934,488

(22) Filed: Sep. 22, 2022

Related U.S. Application Data
(63) Continuation of application No. PCT/KR2021/005205, filed on Apr. 23, 2021.

(30) Foreign Application Priority Data
Apr. 29, 2020 (KR) 10-2020-0052633

(51) Int. Cl. A61B 10/00; A61B 10/02; A61B 10/04; A61B 34/30

(52) U.S. Cl. CPC A61B 10/0041; A61B 10/0266; A61B 10/04; A61B 34/30; A61B 2010/0208

(57) ABSTRACT

The present invention relates to a device for incising a breast specimen, comprising: a fixing unit of which a portion is inserted into an incision window and which fixes a breast specimen to correspond to the entire size of the breast specimen; an incising unit for incising the fixed breast specimen; and an incising control unit adjusting the position and the incising angle of the incising unit.

### CROSS-REFERENCE TO RELATED APPLICATION

The present application is a continuation of International Patent Application No. PCT/KR2021/005205, filed on Apr. 23, 2021, which is based upon and claims the benefit of priority to Korean Patent Application No. 10-2020-0052633, filed on Apr. 29, 2020. The disclosures of the above-listed applications are hereby incorporated by reference herein in their entirety.

### BACKGROUND

### 1. Technical Field

The present invention relates to a device and a method for incising a breast specimen.

### 2. Description of Related Art

Malignant breast tumors affect women more than men, and are tumors, among all types of tumors, that have the greatest number of deaths caused thereby.

In order to remove breast tumors, robotic endoscope (laparoscope) surgery including robotic breast surgery and minimally invasive endoscopic breast surgery (hereinafter, referred to as a robotic endoscopic surgery) has recently been in the spotlight. In order to perform robotic endoscopic surgery, a small incision window is formed so that a surgical scar is not greatly generated on the breast skin, and then a trocar in which several surgical instruments can be inserted is inserted into the small incision window. In this instance, a surgical robot or endoscopic arm and a robot or endoscopy camera are inserted through the trocar to separate the breast tumor within normal parenchyma from the peripheral tissues including the overlying skin, nipple-areolar complex, and pectoralis muscle.

However, it may be difficult to remove a specimen through the small incision window performed by the robotic (or endoscopic) breast surgery since breast parenchyma is harder than other tissues. incisionAccordingly, the conventional breast specimen retrieval (or harvesting, or removing) method requires a larger breast skin incision than the small incision window in order to remove the entire breast specimen. Furthermore, the breast tissue is difficult to be sharply incised by surgical scissors due to its hard texture of the connective tissue. If the tumor surrounding normal breast parenchyma is not properly removed, it is possible to increase the risk of recurrence of cancer due to cells remaining on the incised portion.

Accordingly, there is a need for an incising tool capable of removing a breast specimen through a small incision window formed by the minimally invasive endoscopic surgery and incising the breast specimen.

### SUMMARY

The present disclosure has been made to solve the above-mentioned problems occurring in the prior art, and in an aspect of the present disclosure, an object of the present disclosure is to provide a device and a method for retrieving a breast specimen, capable of incising a breast specimen into a predetermined size without disintegrating the breast specimen into microtissues.

Another object of the present disclosure is to provide a device and a method for incising a breast specimen, capable of incising a breast specimen multiple times with a narrow incision window.

To accomplish the above-mentioned objects, according to an aspect of the present invention, there is provided a breast specimen incising device including: a fixing unit of which a portion is inserted into an incision window and which fixes a breast specimen to correspond to the entire size of the breast specimen; an incising unit for incising the fixed breast specimen; and an incising control unit adjusting the position and the incising angle of the incising unit.

In another aspect of the present invention, there is provided a breast specimen incising method including the operations of: inserting a portion of a fixing unit corresponding to the size of a breast specimen into an incision window to fix the breast specimen; incising the fixed breast specimen with an incising unit; and adjusting the position and the incising angle of the incising unit by an incising control unit.

### BRIEF DESCRIPTION OF THE DRAWINGS

FIG. 1 is a cross-sectional view schematically illustrating a breast specimen incising device according to an embodiment of the present invention.

FIG. 2 is a flow chart schematically illustrating a breast specimen incising method according to an embodiment of the present invention.

FIGS. 3A, 4A, 5A, and 6A are cross-sectional views sequentially showing that a breast specimen is incised by the breast specimen incising device according to an embodiment of the present invention.

FIGS. 3B, 4B, 5B, and 6B are cross-sectional views schematically illustrating cross sections of breast specimens respectively corresponding to FIGS. 3A, 4A, 5A, and 6A.

### DETAILED DESCRIPTION

Advantages and features of the present disclosure and methods accomplishing the advantages and features will become apparent from the following detailed description of exemplary embodiments with reference to the accompanying drawings. However, the present disclosure is not limited to exemplary embodiment disclosed herein but will be implemented in various forms. The exemplary embodiments are provided so that the present disclosure is completely disclosed, and a person of ordinary skilled in the art can fully understand the scope of the present disclosure. Therefore, the present disclosure will be defined only by the scope of the appended claims.

Terms used in the specification are used to describe specific embodiments of the present disclosure and are not intended to limit the scope of the present disclosure. In the specification, terms in singular form may include plural forms unless otherwise specified. It should be also understood that the terms of ‘include’ or ‘have’ in the specification are used to mean that there is no intent to exclude the existence or addition of other components besides components described in the specification. In the detailed description, the same reference numbers of the drawings refer to the same or equivalent parts of the present disclosure, and the term “and/or” is understood to include a combination of one or more of components described above. It will be understood that terms, such as “first” or “second” may be used in the specification to describe various components but are not restricted to the above terms. The terms may be used to discriminate one component from another component. Therefore, of course, the first component may be termed the second component within the scope of the present disclosure.

Unless otherwise defined, all terms (including technical and scientific terms) used herein have the same meaning as commonly understood by those skilled in the technical field to which the present disclosure pertains. It will be further understood that terms, such as those defined in commonly used dictionaries, should not be interpreted in an idealized or overly formal sense unless expressly so defined herein.

Hereinafter, preferred embodiments of the present disclosure will be described in detail with reference to the accompanying drawings.

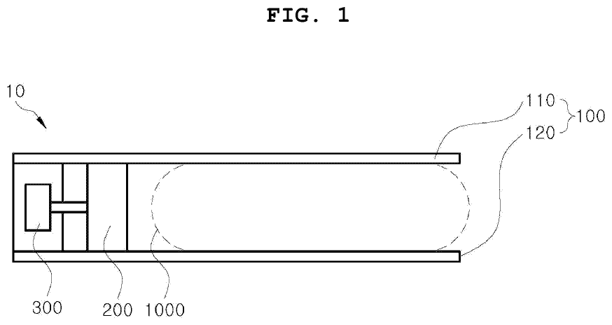

FIG. 1 is a cross-sectional view schematically illustrating a breast specimen incising device according to an embodiment of the present invention.

Referring to FIG. 1, the breast specimen incising device 10 according to the embodiment of the present invention includes a fixing unit 100, an incising unit 200, and an incising control unit 300. FIG. 1 illustrates the breast specimen incising device 10, and the breast specimen incising device 10 is not specifically limited in shape if including the fixing unit 100, the incising unit 200, and the incising control unit 300.

According to the embodiment of the present invention, a portion of the breast specimen incising device 10 is inserted into an incision window CH, and then, a breast specimen 1000 is incised multiple times so that the breast specimen 1000 can be discharged through the incision window CH. More specifically, according to an embodiment of the present invention, after a portion of the breast specimen incising device 10 is inserted into the incision window CH, the breast specimen 1000 is incised multiple times till being discharged from the incision window CH.

A portion of the fixing portion 100 is inserted into the incision window CH. The incision window CH may be a window into which a trocar or a single port is inserted when the robotic surgery is carried out.

Before a portion of the fixing unit 100 is inserted into the incision window CH, the incision window CH is formed in the breast 3000. The incision window CH has a size of 20 to 60 mm. However, the size of the incision window CH may vary depending on the breast size of a patient.

Before a portion of the fixing unit 100 is inserted into the incision window CH, a pouch 2000 is inserted into the incision window CH. The pouch 2000 is inserted into the incision window CH in a state in which the breast specimen 1000 is included therein.

The fixing unit 100 corresponds to the size of the breast specimen 1000. The size of the breast specimen 1000 is, for example, 100 to 1000 grams. The fixing unit 100 fixes the breast specimen 1000.

The fixing unit 100 includes an upper fixing unit 110 and a lower fixing unit 120. For example, in order to fix the breast specimen 1000 with the fixing unit 100, an interval between the upper fixing unit 110 and the lower fixing unit 120 is greater than the size of the breast specimen 1000, and then, is smaller than the size of the breast specimen 1000 so as to fix the breast specimen 1000.

The width of the fixing unit 100, namely, the shortest interval between the upper fixing unit 110 and the lower fixing unit 120, is formed enough to grasp the breast specimen 1000.

The length of the fixing unit 100 inserted into the incision window CH is formed enough to completely incise the breast specimen 1000 across the entire breast specimen 1000. That is, the length of the fixing unit 100 inserted into the incision window CH is adjusted according to the size of the breast specimen 1000, for example, the diameter.

The fixing unit 100 is not specifically limited in shape, for example, a tube shape, if the fixing unit 100 can fix the breast specimen 1000. The upper fixing unit 110 fixes the upper part of the breast specimen 1000. The lower fixing unit 120 fixes the lower part of the breast specimen 1000.

The incising unit 200 cuts the fixed breast specimen 1000. The incising unit 200 is not inserted into the incision window CH.

The number of cuts that the incising unit 200 cuts the fixed breast specimen 1000 may be determined during a surgical operation while checking whether the incised breast specimen can be removed through the incision window CH while directly performing incising, or may be roughly determined through ultrasound imaging, mammography, breast MRI, or body examination performed before the surgery.

The incising unit 200 can move along the fixing unit 100. For example, the incising unit 200 receives electric power from the outside in order to incise the breast specimen 1000.

For example, the incising unit 200 may be an electric knife or a manual knife for incising the breast specimen 1000 by receiving power from the outside while moving along the fixing unit 100.

The incising control unit 300 adjusts the position and the incising angle of the incising unit 200. The incising control unit 300 adjusts the position and the incising angle of the incising unit 200 inserted into the incision window CH.

For example, the incising control unit 300 rotates inside the incision window CH to adjust incising of the breast specimen 1000.

The incising unit 200 is, for example, in the form of a straight line CL1, and cuts the fixed breast specimen 1000, and then, the incising control unit 300 adjusts the position and the incising angle of the incising unit 200. The incising unit 200 which is adjusted in position and incising angle can incise the breast specimen 1000 fixed in the form of a curved line CL2 while rotating. The size of the incised breast specimen 1000 is for example, in a range from 100 to 200 grams (g).

Although not shown, the breast specimen incising device 10 according to the embodiment of the present invention further includes a driving unit. The driving unit can adjust the moving speed and the moving distance of the incising unit 200.

According to an embodiment of the present invention, the breast specimen incising device 10 is inserted into the incision window CH used for a robotic surgery, fixes the breast specimen, and cuts the breast specimen in a linear shape and a curved shape. Therefore, the breast specimen incising device 10 according to an embodiment of the present invention can discharge the breast specimen out of the breast after incising the breast specimen inside the small incision window CH.

The breast specimen incising device 10 according to the embodiment of the present invention can incise the breast specimen into a predetermined size without disintegrating the breast specimen into microtissues. In addition, the breast specimen incising device 10 according to the embodiment of the present invention can incise the breast specimen several times with the one narrow incision window CH.

Hereinafter, a breast specimen incising method according to an embodiment of the present invention will be described. Hereinafter, differences between the breast specimen incising device and the breast specimen incising method will be described in detail, and undescribed parts can be the same as the breast specimen incising device for incising the breast specimen 1000.

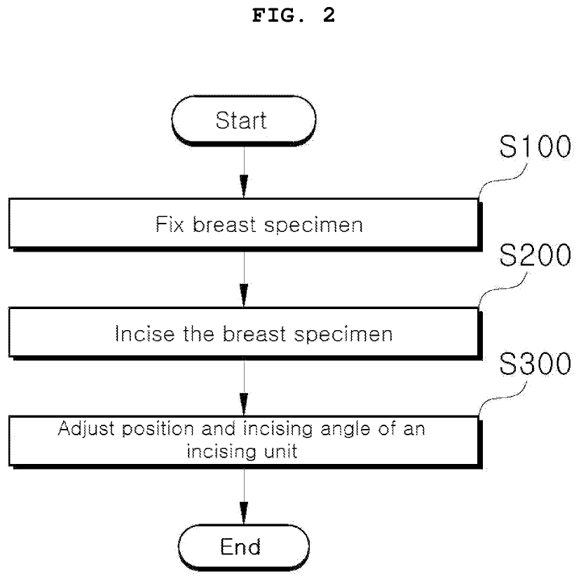

FIG. 2 is a flow chart schematically illustrating the breast specimen incising method according to an embodiment of the present invention.

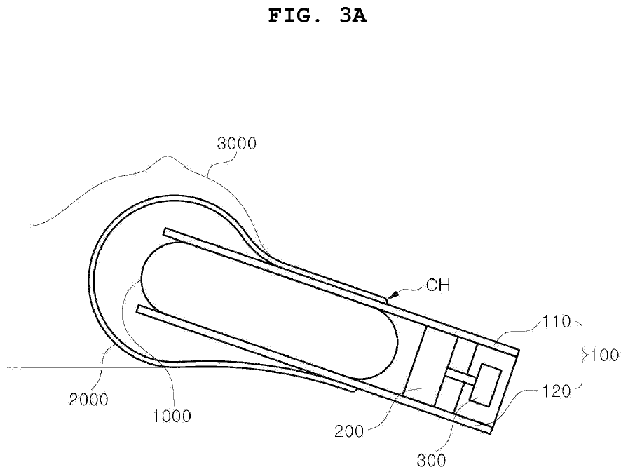

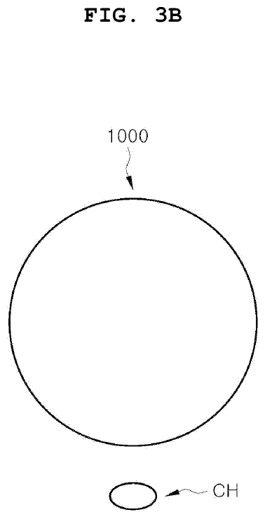

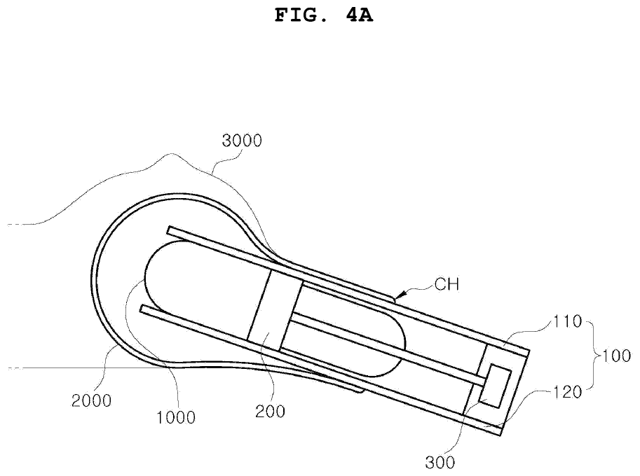

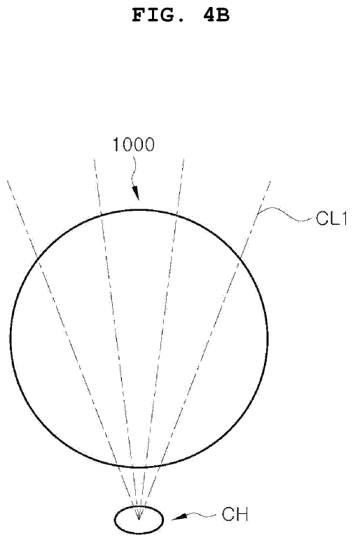

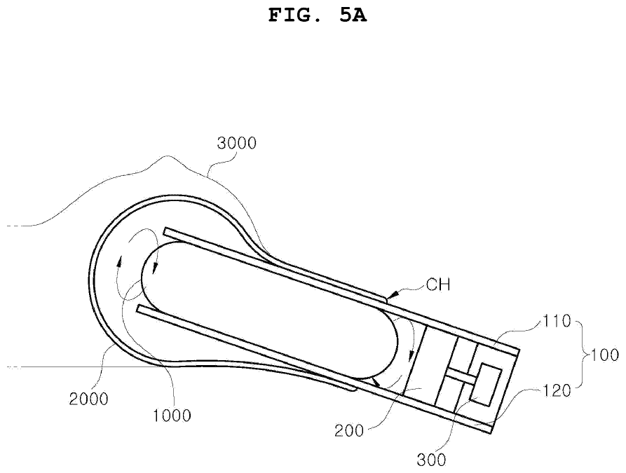

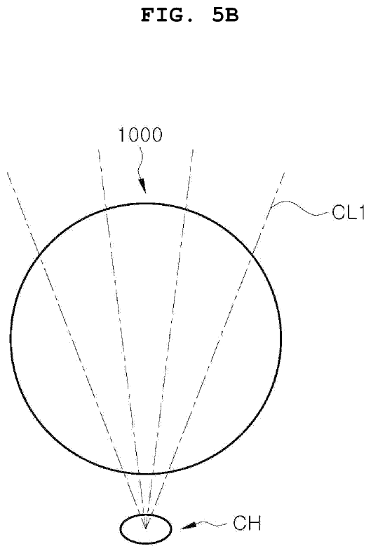

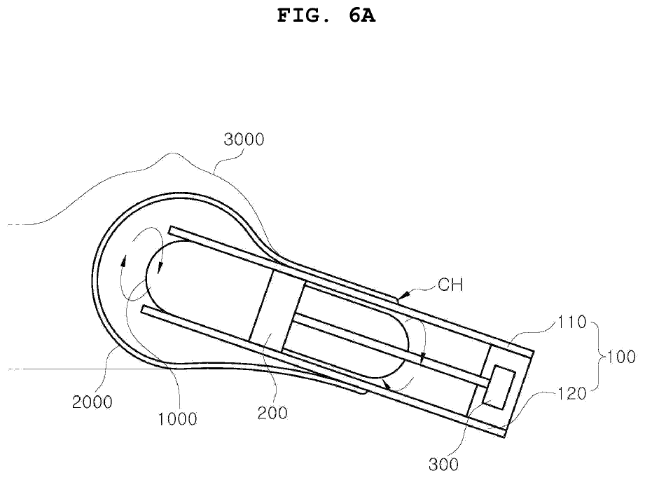

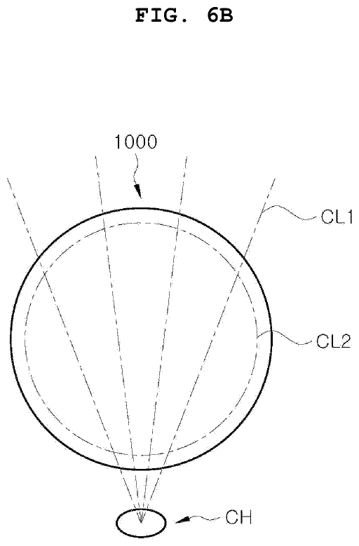

FIGS. 3A, 4A, 5A, and 6A are cross-sectional views sequentially showing that the breast specimen 1000 is cut by the breast specimen incising device 10 according to the embodiment of the present invention. FIGS. 3B, 4B, 5B, and 6B are cross-sectional views schematically illustrating cross sections of breast specimens 1000 respectively corresponding to FIGS. 3A, 4A, 5A, and 6A.

Referring to FIGS. 1B to 6B, the breast specimen incising method according to an embodiment of the present invention includes: a fixing operation (S100) of inserting a portion of a fixing unit 100 corresponding to the size of a breast specimen 1000 into an incision window CH to fix the breast specimen 1000; an incising operation (S200) of incising the fixed breast specimen 1000 by an incising unit 200; and an incising control operation (S300) of adjusting the position and the incising angle of the incising unit 200 by an incising control unit 300.

Through the breast specimen incising method according to an embodiment of the present invention, a portion of the breast specimen incising device 10 is inserted into the incision window CH, and then, the breast specimen incising device cuts the breast specimen 1000 several times so as to discharge the breast specimen 1000 through the incision window CH. More specifically, the breast specimen incising method according to an embodiment of the present invention can incise the breast specimen 1000 multiple times until a portion of the breast specimen incising device 10 is discharged through the incision window CH after being inserted into the incision window CH.

Referring to FIGS. 1, 2, 3A and 3B, a portion of the fixing unit 100 corresponding to the size of the breast specimen 1000 is inserted into the incision window CH to fix the breast specimen 1000 (S100). The portion of the fixing unit 100 is inserted into the incision window CH. The incision window CH may be a window to insert a trocar or a single port for the robotic surgery. The fixing unit 100 corresponds to the size of the breast specimen 1000. The size of the breast specimen 1000, is, for example, in a range of 100 to 1000 grams (g). The fixing unit 100 fixes the breast specimen 1000.

Before a portion of the fixing unit 100 is inserted into an incision window CH, the incision window CH is formed in the breast 3000. The size of the incision window CH is for example in the range of 20 to 60 mm.

Before a portion of the fixing unit 100 is inserted into the incision window CH, a pouch 2000 may be inserted into the incision window CH. The pouch 2000 is inserted into the incision window CH so that the breast specimen 1000 is included therein.

The fixing operation S100 includes the operations of: fixing the upper portion of the breast specimen 1000 to the upper fixing portion 110; and fixing the lower portion of the breast specimen 1000 to the lower fixing portion 120.

In the fixing operation (S100), for example, in order to fix the breast specimen 1000 with the fixing unit 100, the width of the upper fixing unit 110 and the lower fixing unit 120 becomes wider than the breast specimen 1000, and then, gets narrower to fix the breast specimen 1000.

The width of the fixing unit 110, for instance, the shortest distance between the upper fixing unit 110 and the lower fixing unit 120, is enough to hold the breast specimen 1000.

The fixing unit 100 inserted into the incision window CH has the length to completely incise across the breast specimen 1000. That is, the length of the fixing unit 100 inserted into the incision window CH can be adjusted according to the size of the breast specimen 1000, for instance, the diameter of the breast specimen 1000.

Referring to FIGS. 1, 2, 4a and 4b, the incising unit 200 cuts the fixed breast specimen 1000 (S200). The incising unit 200 can incise the fixed breast specimen 1000. The incising unit 200 is not inserted into the incision window CH. For instance, the incising unit 200 can incise the breast specimen 1000 in a straight line form CL1.

The incising unit 200 can move along the fixing unit 100. The incising unit 200 may, for example, incise the breast specimen 1000 by electric power supplied from the outside.

The incising unit 200 may be, for example, an electronic knife which receives electric power from the outside while moving along the fixing unit 100 to incise the breast specimen 1000.

Referring to FIGS. 1, 2, 5A and 5B, the incising control unit 300 adjusts the position and the incising angle of the incising unit 200 (S300). The incising control unit 300 may adjust the position and the incising angle of the incising unit 200 inserted into the incision window CH.

For example, in the incising control operation (S300), the incising unit 200 rotates in the incision window CH to adjust incising of the breast specimen 1000.

The incising control unit 300, is for example, in the form of a straight line, and can adjust the position and the incising angle of the incising unit 200 after incising the fixed breast specimen 1000.

Referring to FIGS. 1, 2, 6A and 6B, the incising unit 200 of which the position and the incising angle are adjusted is rotated to incise the fixed breast specimen 1000 in a curved form (CL2). The size of the incised breast specimen 1000 is, for example, in the range of 100 g to 200 g.

The breast specimen incising method according to an embodiment of the present invention further includes an operation of adjusting a moving speed and a moving distance of the incising unit 200. Although not shown, for example, the moving speed and moving distance of the incising unit 200 may be controlled by the driving unit.

The breast specimen incising method according to the embodiment of the present invention can insert a breast specimen into the incision window CH used for the robotic surgery, fix the breast specimen, and cut the breast specimen into a straight form and a curved form, thereby incising the breast specimen inside the incision window CH and discharging the breast specimen to the outside of the breast.

The breast specimen incising method according to the embodiment of the present invention can incise the breast specimen into a predetermined size without disintegrating the breast specimen into microtissues. In addition, the breast specimen incising method according to the embodiment of the present invention can incise the breast specimen multiple times with one narrow incision window.

The above description is only exemplary, and it will be understood by those skilled in the art that the disclosure may be embodied in other concrete forms without changing the technological scope and essential features. Therefore, the above-described embodiments should be considered only as examples in all aspects and not for purposes of limitation.

### Advantageous Effects

As described above, the device and the method for incising a breast specimen can incise a breast specimen into a predetermined size without disintegrating the breast specimen into microtissues.

According to the present disclosure, the device and the method for incising a breast specimen can incise breast specimens multiple times through one narrow incision window.

1. A breast specimen incising device comprising:
a fixing unit of which a portion is inserted into an incision window and which fixes a breast specimen to correspond to the entire size of the breast specimen;
an incising unit for incising the fixed breast specimen; and
an incising control unit adjusting the position and the incising angle of the incising unit.

2. The breast specimen incising device according to claim 1, wherein the incision window is a window through which a trocar or a single port is inserted when a robotic endoscopic surgery is carried out.

3. The breast specimen incising device according to claim 1, wherein the fixing unit comprises:
an upper fixing unit fixing an upper portion of the breast specimen; and
a lower fixing unit fixing a lower portion of the breast specimen.

4. The breast specimen incising device according to claim 1, further comprising:
a driving unit adjusting a moving speed and a moving distance of the incising unit.

5. The breast specimen incising device according to claim 1, wherein the incising control unit adjusts the incising unit to rotate inside the incision window to incise the breast specimen.

6. The breast specimen incising device according to claim 1, wherein the incising unit cuts the breast specimen multiple times.

7. A breast specimen incising method comprising the operations of:
inserting a portion of a fixing unit corresponding to the size of a breast specimen into an incision window to fix the breast specimen;
incising the fixed breast specimen with an incising unit; and
adjusting the position and the incising angle of the incising unit by an incising control unit.

8. The breast specimen incising method according to claim 7, wherein the incision window is a window through which a trocar or a single port is inserted when a robotic surgery is carried out.

9. The breast specimen incising method according to claim 7, wherein the fixing operation comprises the operations of:
fixing an upper portion of the breast specimen; and
fixing a lower portion of the breast specimen.

10. The breast specimen incising method according to claim 7, further comprising the operation of:
adjusting a moving speed and a moving distance of the incising unit.

11. The breast specimen incising method according to claim 7, wherein in the incising control operation, the incising unit rotates inside the incision window to incise the breast specimen.

12. The breast specimen incising method according to claim 7, wherein in the incising operation, the breast specimen is incised multiple times.

13. The breast specimen incising method according to claim 7, Before a portion of the fixing unit is inserted into the incision window, a pouch is inserted into the incision window.
